(19) United States
(12) Patent Application Publication
(10) Pub. No.: US 2015/0147378 A1
(43) Pub. Date: May 28, 2015

(54) COPOLYMER-XEROGEL NANOCOMPOSITES USEFUL FOR DRUG DELIVERY

(71) Applicants: RUTGERS, THE STATE UNIVERSITY OF NEW JERSY, New Burnswick, NJ (US); THE TRUSTEES OF THE UNIVERSITY OF PENNSYLVANIA, Philadelpha, PA (US)

(72) Inventors: David Devore, Fort Sam Houston, TX (US); Paul Ducheyne, Rosemount, PA (US); Marius C. Costache, Lexington, MA (US)

(21) Appl. No.: 14/402,557

(22) PCT Filed: May 21, 2013
(86) PCT No.: PCT/US2013/041996

§ 371 Date: Nov. 20, 2014

Related U.S. Application Data
(63) Continuation-in-part of application No. 12/476,009, filed on Jun. 1, 2009.
(60) Provisional application No. 61/649,643, filed on May 21, 2012.
(60) Provisional application No. 61/057,642, filed on May 30, 2008.

(51) Int. Cl. A61L 31/16; A61L 27/54; A61L 27/44; A61L 27/18; A61L 27/58; A61L 31/12; A61L 31/06; A61L 31/02; A61L 31/14; A61L 26/00; A61L 27/02

(52) U.S. Cl. CPC A61L 31/16; A61L 26/0066; A61L 26/0095; A61L 26/0019; A61L 26/0004; A61L 27/54; A61L 27/446; A61L 27/18; A61L 27/02; A61L 31/128; A61L 31/06; A61L 31/028; A61L 31/148; A61L 27/58; A61L 26/009; A61L 2300/406; A61L 2300/402; A61L 2300/604
USPC 424/422; 514/254.11; 514/330; 424/489

(57) ABSTRACT

Biocompatible, biodegradable copolymer-xerogel nanocomposites contain a biodegradable, biocompatible copolymer, silica nanoparticles and one or more therapeutic agents, that are useful for wound dressings, medical devices and drug delivery applications.

### CROSS-REFERENCE TO RELATED APPLICATIONS

This application is a continuation-in-part under 35 U.S.C §120 of U.S. patent application Ser. No. 12/476,009, filed on Jun. 1, 2009, which claims the benefit of priority under 35 U.S.C §119(c) of U.S. Provisional Application No. 61/057,642, filed on May 30, 2008. This application also claims the benefit of priority under 35 U.S.C §119(e) of U.S. Provisional Application No. 61/649,643, filed on May 21, 2012. The disclosures of all of the above documents are incorporated herein by reference in their entireties.

### STATEMENT OF GOVERNMENT RIGHTS

Research leading to the disclosed inventions was funded, in part, by the United States Army, CDMRP grant number W81XWH-07-1-0438. Accordingly, the United States Government has certain rights in the invention.

### FIELD OF TECHNOLOGY

The present invention is directed to nanocomposites useful for drug delivery applications which provide controlled delivery of therapeutic agents, including drug “depots”, wound dressings, stents and tissue scaffolds.

### BACKGROUND OF THE INVENTION

There is an enormous unmet need for biomaterials than can provide effective local delivery of therapeutic agents with controlled release kinetics. These biomaterials must be able to meet challenging requirements for both mechanical and biological functionality in such critical applications as implantable drug delivery depots, wound dressings and stents. The challenge is particularly acute for hydrophobic drugs with limited aqueous solubility that limits formulation concentrations and hence limits drug diffusion to target substrates such as pathogenic biofilm infections in chronic wounds and on orthopedic fracture fixation devices, peripheral nerves involved in chronic pain syndromes, solid tumors or cardiovascular stents experiencing restenosis.

Biodegradable composites that combine the processability and viscoelasticity of biodegradable organic polymers with the mechanical strength of biodegradable ceramic filler materials offer significant potential to meet these biomaterial performance requirements when, as is often the case, the properties of polymers or ceramics alone are inadequate. High mechanical strength is imparted to composites through effective load transfer between the continuous polymer matrix and the discontinuous inorganic particles. This requires effective interfacial bonding, either physical or covalent, between the ceramic and polymeric components. The interfacial properties of polymer-inorganic composites also exert a strong influence on other properties including gas permeability, water uptake, drug release kinetics and cellular responses. Both the physical and chemical properties of biomaterials can strongly affect the performance of and biological responses to drug delivery devices, wound dressings and tissue engineering scaffolds. When the polymers used to form the composites are biodegradable, the biodegradation rates are significantly altered by the presence of the inorganic components, their concentration in the composite matrix and whether they are physically or covalently bonded to the organic polymer components.

Early treatment of bodily wounds is generally limited to hemostasis and administration of pain medication. For example, for severe battlefield wounds, the initial treatment consists of applying hemostatic agents such as chitosan bandages and Quick-Clot™ zeolite. Wound dressings being deployed on the battlefield, however, are not designed to deliver pain medication. Existing injectable hydrogels, such as Durect's SABER™ system for delivery of bupivacaine, are not designed for battlefield applications because they cannot withstand the conditions that occur during transport of patients to medical facilities. Further, traditional anesthetic delivery systems such as direct injection, epidural catheters, and intra-articular indwelling catheters are not designed or convenient for battlefield applications. These modalities of local delivery of analgesics are not designed to withstand the conditions present during the transport of patients, have limited efficacy, have potential adverse clinical complications, and require highly trained medical personnel. As a result, on-the-field pain treatment of wounds is usually delivered in the form of systemic morphine injections, which have numerous unwanted and serious side effects. Similarly, in the case of wound infections infections, effective treatment is exacerbated by multi-drug resistant strains of microrganisms (MDRO's) such as methicillin-resistant Staphylococcus aureus (MRSA), Pseudomnonas aeruginosa, Enterococcus faecium, Escherichia coli, Klebsiella pneumoniae, Enterobacter species, and Acinetobacter baumanni. Topical delivery of antimicrobials and other therapeutic agents is advantageous because systemic toxicity is avoided and high local concentrations can be achieved that are often necessary to eradicate drug-resistant microbial biofilms, particularly in cases where systemic delivery resulting from ischemia at wound sites can limit other parenteral or oral drug delivery routes. For chronic open wounds, it is widely recognized that a moist environment promotes wound healing and this is generally accomplished in the clinic by the application of occlusive polymeric hydrogel wound dressings.

Severe combat wounds, particularly blast wounds resulting from explosive devices, involve substantial tissue damage that produces sustained and often intense levels of pain throughout and beyond the early tissue healing process. If the pain is left untreated, the pain signals can be imprinted in the central nervous system, resulting in chronic pain. Continuous peripheral nerve block by local delivery of anesthetics immediately following trauma or surgical procedures has been suggested to have potential to prevent chronic pain, including syndromes such as phantom limb pain. Thus, it is highly desirable to provide controlled delivery of local anesthetics directly to a wound.

In some instances, severe combat wounds, particularly blast wounds, also result in compartment syndrome. Compartment syndrome occurs when elevated intramuscular pressure decreases vascular perfusion of a muscle compartment to a point no longer sufficient to maintain viability of the muscle and neural tissue contained within the compartment. Compartment syndromes can result from multiple types of injuries including orthopedic (traumatic), vascular, iatrogenic, and soft tissue. Blast injuries now seem to fall in this category as well. In some cases the blast injury can only be part of soft tissue injury or it can be a combination of the other etiologies including components of orthopedic, vascular and/or soft tissue. More recently with the increasing number of casualties from blast injury, it is hypothesized that the blast causes a direct injury to the muscle that results in swelling and a secondary compartment syndrome.

Generally, in compartment syndromes, there is an increasing pressure within a tissue compartment that needs to be released as soon as possible, often within 4 to 6 hours. Compartment syndromes must be treated early in the time line of wound care that begins at the battlefield and ends in the hospital. If a compartment syndrome is not diagnosed early, a Volkmann contracture can occur with massive loss of all tissues within the compartment. Untreated compartment syndrome can lead to tissue necrosis, permanent functional impairment, renal failure, and death. However, the standard diagnosis of compartment syndrome by clinical signs—including myoneural pain with passive stretch, paresthesia, and paresis—is often masked by other injuries in patients with blast injuries who suffer polytrauma.

The treatment of compartment syndrome requires the release of the fascia that enclose the compartments within the first three to six hours to prevent irreversible injury to the nerves and muscles. Once the compartments are released the open wounds are treated with dressings to prevent infection and protect the wound. In some cases a specialized Vacuum Assisted Closure System is used to cover and protect the wound. The open wounds are then kept dressed for 48 to 72 hours until the patients are returned to the operating room for a second look to allow further debridement of non-viable muscle tissue if indicated. Fasciotomies, however, extend hospital stays and change a closed injury to an open injury, greatly increasing the chance of infection. Further, there is some debate about the criterion for performing a fasciotomy, with recommendations varying from prophylactic fasciotomy at normal pressure to finding a pressure from 30 mm Hg to 45 mm Hg.

It has been suggested that impeding the early cellular events leading to ischemia and pressure build up in the compartment can be the first line of defense. Thus, it would be desirable to provide controlled delivery of therapeutic agents to prevent the late-stage problems of compartment syndrome and initiate regeneration of healthy tissue.

There remains a great need for materials for the treatment of wounds that effect the controlled release of pharmaceutically active molecules. Controlled release focuses on delivering biologically active agents locally over extended time periods. The site specificity of the delivery reduces the potential side effects that can be associated with general administration of drugs through oral or parenteral therapy. Prevalent mechanisms for the delivery of biological agents by controlled release devices are either resorption of the drug carrier material or diffusion, The resorption of these devices can, however, cause an inflammatory tissue response which interferes with the treatment sought for with the biomolecules.

### BRIEF SUMMARY OF THE INVENTION

It has now been discovered that highly tunable mechanical and controlled drug delivery properties are accessible with novel biodegradable nanocomposites prepared by non-covalent binding of silica xerogels and biodegradable, biocompatible polymers.

Further, it has also been discovered that sustained controlled release of clinically significant drugs, including antibiotics and local anaesthetics, can be obtained from these nanocomposites. Such nanocomposites are thus attractive bioactive biomaterials for applications including wound dressings, tissue engineering applications, cardiovascular stents and nerve guides or conduits. One embodiment of the present invention is directed to a copolymer-xerogel nanocomposite, comprising a biodegradable, biocompatible copolymer, silica nanoparticles and one or more therapeutic agents.

A further embodiment of the present invention is directed to a method of forming a therapeutic agent-loaded copolymer-xerogel nanocomposite, comprising the steps of:

(a) providing a silica sol;

(b) adding said silica sol to a poly(desaminotyrosyl tyrosine ester-co-PEG carbonate) to form a mixture;

(c) adding one or more therapeutic agents to said mixture to form a drug mixture; and

(d) removing the solvents from said drug mixture to form the therapeutic agent-loaded copolymer-xerogel nanocomposite.

Another embodiment of the present invention provides drug depots, wound dressings, tissue scaffolds, cardiovascular stents and nerve guides containing the copolymer-xerogel nanocomposites. The devices are adapted to bind and release therapeutic agents, thereby providing controlled delivery of the therapeutic agents for healthcare applications.

One embodiment of the invention is directed to a copolymer-xerogel nanocomposite, comprising a biodegradable, biocompatible copolymer, silica nanoparticles and one or more therapeutic agents. In a preferred embodiment, the biodegradable, biocompatible copolymer has a molecular weight greater than 20,000 Daltons. In a further embodiment, the therapeutic agent comprises a compound selected from the group consisting of antibiotics, local anesthetics, and combinations of two or more thereof. In one embodiment, the therapeutic agent is rifampicin and/or bupivacaine.

In another embodiment of the above copolymer-xerogel nanocomposite, the biodegradable copolymer comprises a copolymer of tyrosine-poly(alkylene glycol)-derived poly(ether carbonate). In one embodiment, the biodegradable copolymer comprises a polycarbonate comprising desaminotyrosyl tyrosine ester and poly(ethylene glycol). In a further embodiment, the above copolymer-xerogel nanocomposite is adapted to provide controlled release of the therapeutic agent(s).

Another embodiment of the invention is directed to a method of forming a therapeutic agent-loaded copolymer-xerogel nanocomposite, comprising:

(a) providing a silica sol;

(b) adding said silica sol to a poly(desaminotyrosyl tyrosine ester-co-PEG carbonate) to form a mixture;

(c) adding one or more therapeutic agents to said mixture to form a drug mixture; and

(d) removing the solvents from said drug mixture to form the therapeutic agent-loaded copolymer-xerogel nanocomposite.

In a further embodiment of the method the therapeutic agent is selected from the group consisting of antibiotics, local anesthetics, and combinations of two or more thereof. In one embodiment the therapeutic agent is rifampicin and/or bupivacaine.

Other embodiments of the invention are directed to:

a drug depot comprising the above nanocomposite;

a wound dressing comprising the above nanocomposite;

a tissue scaffold comprising the above nanocomposite; and

a cardiovascular stent comprising the above nanocomposite.

In one embodiment of the cardiovascular stent, the one or more therapeutic agents of the nanocomposite are selected from the group consisting of drugs which control restenosis. In a specific embodiment the cardiovascular stent is adapted to provide controlled release of the drugs which control restenosis. In one embodiment the therapeutic agents are selected from the group consisting of everolimus, sirolimus (rapamycin), zotarolimus, and paclitaxel.

Another embodiment of the invention is directed to a method of treating a wound comprising applying to the wound a copolymer-xerogel nanocomposite, comprising a biodegradable, biocompatible copolymer, silica nanoparticles and one or more therapeutic agents. In a further embodiment, the method is adapted to provide controlled release of the therapeutic agent(s). In one embodiment of the method of treating a wound, the therapeutic agent is selected from the group consisting of antibiotics, local anesthetics, and combinations of two or more thereof. In a specific embodiment said therapeutic agent is rifampicin and/or bupivacaine.

Another embodiment of the invention is directed to a hollow tube nerve guide comprising the above nanocomposite. In one embodiment, the one or more therapeutic agents of the nanocomposite are selected from the group consisting of neurotrophic factors. In one embodiment the hollow tube nerve guide is adapted to provide controlled release of the neurotrophic factors.

The present invention can be understood more readily by reference to the following detailed description taken in connection with the accompanying figures and examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

### BRIEF DESCRIPTION OF THE DRAWINGS

FIG. 1 shows the chemical structure of tyrosine-PEG-derived poly(ether carbonate). Adjustable parameters are: x, the mole fraction of the desaminotyrosyl tyrosine-derived monomer (DTR) (x is fixed at 90% in one embodiment) and y, the mole fraction of PEG (y is fixed at 10% in one embodiment); R, the pendent alkyl chain length (i.e. the monomer is “DTE” when the pendent group is ethyl, or “DTO” when it is octyl); and the PEG molecular weight is fixed at 1,000 Daltons (degree of polymerization=23) in one embodiment.

FIG. 2 shows a nanocomposite film of the invention (O0010/N25) containing 25% silica xerogel before (A) and after (B) heating the film at 700CC to burn off the poly(DTO-10% PEGcarbonate) copolymer matrix.

FIG. 3 shows TEM images of poly(DTE-10% PEG carbonate) nanocomposites at 3% (A) and 10% (B) silica xerogel loading.

FIG. 4 shows the glass transition temperatures of composites as a function of silica xerogel particle size distributions. E0010(1k) (first bar) is poly(DTE-10% PEG 1k)carbonate. Composites of E0010(1K) with micron-scale silica xerogel composites (gray bars) and E0010/N30 nanocomposite (black bar) are all at a fixed silica xerogel content of 30% (w/w).

FIG. 5 displays the effect of silica xerogel content on the glass transition temperature, Tg, of nanocomposites. Copolymer, poly(DTE-10% PEG1k) carbonate and nanocomposites.

FIG. 6 displays the effect of particle size in the composite formulations on the Young's modulus. The xerogel content was fixed at 30 wt %. Polymer is poly(DTE-10% PEG1k) carbonate (E0010).

FIG. 7 shows the stress-elongation curves change with the fraction of silica in the E0010 nanocomposites. Low amounts of silica toughen the polymer (A), while higher loadings make it stronger but more brittle (B).

FIG. 8 displays the equilibrium water content for nanocomposites are a function of silica xerogel concentration. Experimental EWC values (black bars) where EWC=(W_{wet}−W_{dry})/W_{dry}; calculated EWC values (open bars) are based on the assumption that only the mass of copolymer present takes up water.

FIG. 9 displays the hydrolytic mass loss of nanocomposites as a function of silica content: ⋄=copolymer (00010); ▴=nanocomposite with 5% xerogel; □=nanocomposite with 25% xerogel; =nanocomposite with 50% xerogel.

FIG. 10 shows a graph of bupivacaine release from copolymer, microcomposite and nanocomposite, versus time: □=copolymer poly(DTO-10% PEG1k) carbonate (O0010). ⋄=microcomposite (O0010/M50) and A nanocomposite (O0010/N50). The microcomposite and nanocomposite contain the same silica loading, 50 wt %, and the bupivacaine content is fixed at 8 wt % for all of the samples.

FIG. 11 shows a graph of the nanocomposite release of rifampicin versus time. Rifampicin loading was 10% (wt:wt) in the poly(DTO-10% PEG1k) carbonate/10% silica (O0010/N10) nanocomposite.

### DETAILED DESCRIPTION OF PREFERRED EMBODIMENTS

The efficacy of prior controlled delivery devices for therapeutic agents is generally limited by the problem of so-called burst release kinetics. The nanocomposites of the present invention reduce or eliminate the burst release, instead providing continuous and constant rates of release of the therapeutic agent that are essential for sustained, effective therapeutic activity. The nanocomposites uniquely combine material and drug delivery properties that are essential for wound dressings and various drug delivery applications.

The inventive nanocomposites are biocompatible (i.e. substantially non-cytotoxic and non-inflammatory), biodegradable, flexible, mechanically robust, and in particular are formable into various devices which are capable of providing continuous controlled release of a wide array of therapeutic agents for a useful period of time. Further, the robust, flexible and formable nature of the nanocomposites enables their use as implanted depots or wound dressings not only in hospitals and civilian uses but also in the far more demanding conditions of military uses such as on a battlefield or field hospital.

The silica nanoparticles are preferably biodegradable silica-based glass nanoparticles. A preferred route of processing the particles is by a sol-gel methodology, although other methods can be used. The polymers are preferably high molecular weight, biocompatible, biodegradable amphiphilic hydrogels comprised of, for example, poly(vinyl alcohol) (PVA); poly(alkylene oxides), including poly(ethylene oxide) (PEO, or PEG); polycaprolactone (PCL); polymers of desaminotyrosyl tyrosine; poly(lactic acid) (PLA), polyglycolic acid (PGA), copolymers of lactic and glycolic acid (PLGA); polysaccharides; peptides; and linear, block or graft copolymers of these. High molecular weight of greater than about 20,000 Daltons for the polymer is preferred for effective mechanical properties of the nanocomposites. Amphiphilic polymer properties are conferred by the presence of one or more hydrophilic monomer units and one or more hydrophobic monomer units. Amphiphilic properties enable sustained, controlled delivery of both hydrophilic and hydrophobic drugs.

Examples of desaminotyrosyl tyrosine polymers include polycarbonates, polyarylates, polyiminiocarbonates, polyethers, polyurethanes, polycarbamates, polythiocarbonates, polycarbonodithionates, polyphosphoesters, polyphosphazines and polythiocarbamates of this monomer family. Polycarbonates, specifically poly(amide carbonates), as well as polyurethanes, polycarbamates, polythiocarbonates, polycarbonodithionates and polythiocarbamates are prepared by the process disclosed by U.S. Pat. No. 5,198,507, the disclosure of which is incorporated by reference. Methods adaptable for use to prepare polyarylate polymers of the present invention are disclosed in U.S. Pat. Nos. 5,317,077 and 5,658,995, the disclosures of which are incorporated herein by reference. Polyesters, specifically poly(ester amides), are prepared by the process disclosed by U.S. Pat. No. 5,216,115, the disclosure of which is incorporated herein by reference.

Polyiminocarbonates are prepared by the process disclosed by U.S. Pat. No. 4,980,449, the disclosure of which is incorporated by reference. Polyethers are prepared by the process disclosed by U.S. Pat. No. 6,602,497, the disclosure of which is incorporated by reference. Polyphosphoesters and polyphosphazines are prepared by the process disclosed in U.S. Pat. No. 5,912,225, the disclosure of which is incorporated by reference. The other phosphate polymers disclosed in U.S. Pat. No. 5,912,225 are likewise suitable for use with the present invention. Desaminotyrosyl tyrosine monomers are prepared according to the methods disclosed by U.S. Pat. No. 5,099,060, the entire disclosure of which is incorporated herein by reference.

Random block copolymers of these polymers with poly(alkylene oxides) can be prepared as described in U.S. Pat. No. 5,658,995, the disclosure of which is incorporated by reference. Radio-opaque versions of the foregoing polymers are prepared according to the methods disclosed by U.S. Pat. No. 6,475,477, the entire disclosure of which is incorporated herein by reference.

The polymers and copolymers can be cross-linked, either by covalent or ionic bonding, to form the hydrogels or to otherwise promote critical performance properties including gelling, fluid adsorption and increased mechanical strength. Versions of these polymers with free pendant carboxylic acid groups available for cross-linking are prepared according to the methods disclosed by U.S. Pat. No. 6,120,491, the entire disclosure of which is incorporated herein by reference. Cross-linked versions of these polymers are prepared according to the methods disclosed by U.S. Pat. No. 7,368,169, the entire disclosure of which is incorporated herein by reference.

The nanocomposites provide controllable binding and release of therapeutic agents, thereby providing controlled delivery of the therapeutic agents for healthcare applications. The polymers and the silica nanoparticles independently can contain therapeutic agents, are independently capable of binding these agents, and can independently release such agents. It is sufficient that either the polymer or the nanoparticles contains therapeutic agents, although both can contain them.

The nanocomposites of nanoparticles in various biocompatible, biodegradable polymers provides a unique matrix that enables better control of the kinetics of delivery of the therapeutic agents than can be attained by either the polymers or nanoparticles alone. In one embodiment, the nanoparticles are embedded in polymers having the form of a film, which enables the use of the outstanding release properties of the nanoparticles in applications where a solid sheet is needed for treatment, such as in wound dressings.

In another embodiment, the polymer-nanoparticle nanocomposite is fabricated for use in the depot delivery of therapeutic agents such as organic drug compounds, genes, oligonucleotides, and proteins, and in wound treatment applications such as for compartment syndrome, chronic and phantom pain treatment, hemostasis and infection control. Thus a wide variety of therapeutic agents including, without limitation, antibiotics, local anesthetics, analgesics, vasodialators, and vasoconstrictors can be so delivered. Furthermore, the nanocomposites can be formulated for pseudo-first order release of one or more therapeutic agents therefrom.

Therefore, according to one embodiment, biodegradable, biocompatible silica-polymeric nanocomposites are provided that control delivery of local anesthetics and antibiotics directly to the wound site to provide pain relief and infection control. The biomaterial nanocomposite films provide sustained treatment of the peripheral nerves located at the wound site with a local anesthetic that functions as a sodium channel blocker to shut down the firing of the afferent axons that carry the pain signals back to the brain. This reduces or eliminates the imprinting process in the central nervous system that is recognized as a key component of chronic pain. Similarly, sustained delivery at the wound site of antimicrobial agents eliminates infections caused by pathogenic biofilms that might otherwise lead to osteomyelitis, non-healing of bone fractures and other serious complications. Further, delivery of pain and antimicrobial medication by the robust formable nanocomposites, beginning on the battlefield or in combat support hospitals or in surgical procedures at veterans and civilian hospitals, leads to reduced morbidity, decreased postoperative narcotic usage, and the attenuation of chronic pain and infection syndromes.

In accordance with another aspect of the invention, there is provided a biocompatible nanocomposite designed to counteract the effects of compartment syndrome of the tissues. Thus the present invention provides nanocomposites of biocompatible polymers and bioresorbable silica-based sol-gels that deliver anti-apoptotic and pro-angiogenic factors to seal damaged cell membranes and thereby repair damaged tissues. The nanocomposites also absorb extracellular fluid within the compartment to reduce hydrostatic pressure and minimize the extent of damaged tissue. These treatments can be used prophylactically to reduce, if not eliminate the need for fasciotomies. When required, the treatments can be used to accelerate healing after fasciotomies.

Another aspect of the invention is directed to a cardiovascular stent comprising the inventive nanocomposite. Preferably for stent applications, the therapeutic agents of the nanocomposite are selected from the group consisting of drugs which control restenosis. These can include agents selected from the group consisting of everolimus, sirolimus (rapamycin), zotarolimus, and paclitaxel. The cardiovascular stents of the invention are adapted to provide controlled release of these drugs, which thereby eliminates, reduces, delays or otherwise controls the restenosis process. This control of restenosis can last from months to years, preferably 6 months to 5 years.

Another aspect of the invention is directed to a hollow tube nerve guide or conduit comprising the inventive nanocomposite. Preferably for nerve guide applications, the therapeutic agents of the nanocomposite are selected from the group consisting of neurotrophic factors. The nerve guides of the invention are adapted to provide controlled release of the neurotrophic factors, thereby stimulating regeneration of nerve tissue.

### Nanocomposite Morphology.

When un-modified inorganic particles are mixed into a polymer matrix without the help of a compatibilizer, the dispersed inorganic phase often tends to agglomerate, which results in opaque films with poor mechanical properties. The present invention avoids this problem by preparing nanocomposites in situ from fully miscible precursors. Tetraethyl orthosilicate (TEOS) is hydrolyzed under acidic catalysis conditions and the condensation reaction is allowed to proceed only so far as to form a sol but not a gel state.

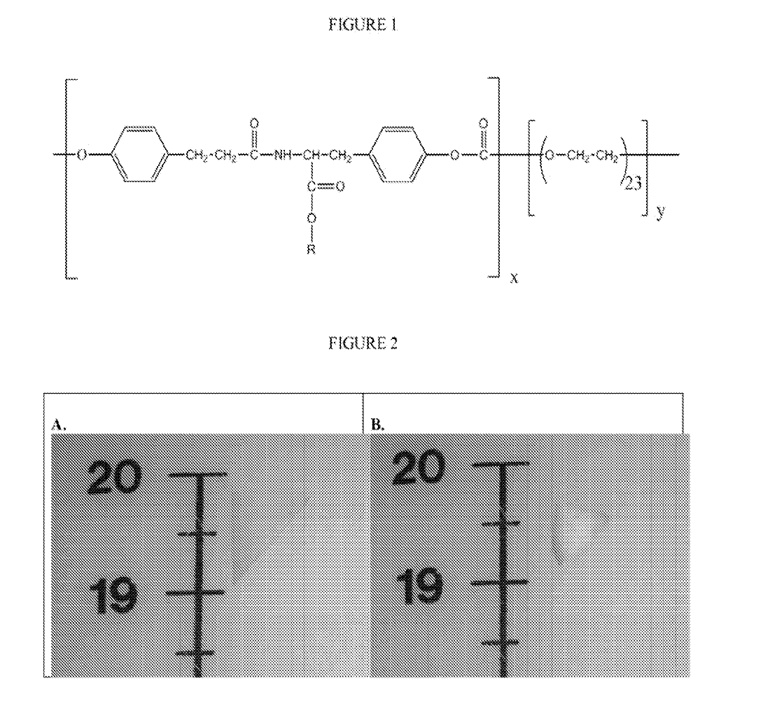

The sol is then mixed vigorously with the copolymer dissolved in acetic acid such that a clear, homogenous solution is obtained. Upon solvent casting and drying this solution, the resulting films are found to be optically transparent (FIG. 2), which is indicative of dispersion of silica at sub-micron particle size. After burning off the copolymer at 700° C., the residual silica maintains the original shape of the film sample, which is further indicative of the uniform dispersion of the silica throughout the copolymer matrix. The observed shrinking of the film is expected given that only 25% of the mass remains after burning the copolymer.

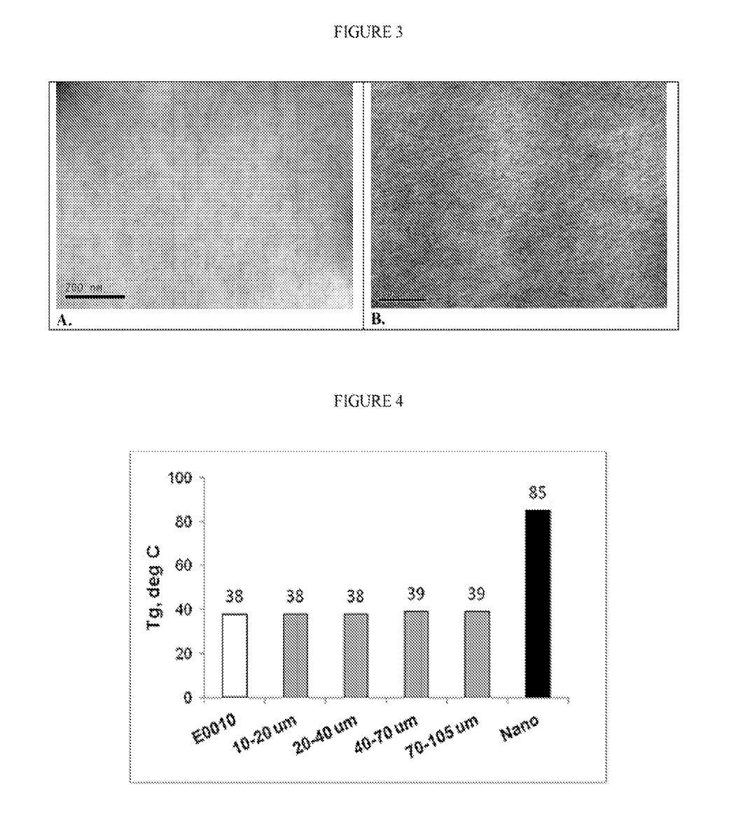

That these films are indeed nanocomposites is confirmed by TEM micrographs of composites containing 3% silica xerogel (E0010/N3) and 10% silica xerogel (E0010/N10) (FIG. 3). In both samples the silica xerogel particles are well below 50 nm diameter and are uniformly distributed throughout the polymer matrix with no micron-scale aggregates present. For E0010/N3, polymer-rich domains (lighter regions) of approximately 50-100 nm in length can be observed, while silica-rich domains are also present (darker regions). As the silica content is increased, the silica distribution remains fairly uniform and the silica xerogel network appears continuous.

### Glass Transition Temperature.

The glass transition temperature, Tg, is a measure of the motion of polymer chain segments and is dependent on chain rigidity, cohesive energy density, polarity, molecular weight and cross-linking between chains. Above the Tg the cooperative movement of a certain number of backbone units is allowed and the polymer chains can slide past each other when a force is applied. For the micron-scale xerogel composites containing 30% silica particles ranging in size from 10 to 105 μm, the Tg's are all 38-39° C., the same as that of the copolymer alone (FIG. 4). This is indicative of minimal perturbation of polymer chain motions by the micron-scale silica particles and hence of weak interfacial interactions between the copolymers and silica particles. For the 30% silica-containing nanocomposite, however, the Tg is 85° C., which is 46° C. higher than that of the copolymer or the micron-scale xerogel particle composites. This is indicative of a significant interfacial interaction between the copolymer chains and the nano-scale silica particles that significantly restricts copolymer chain segment mobility.

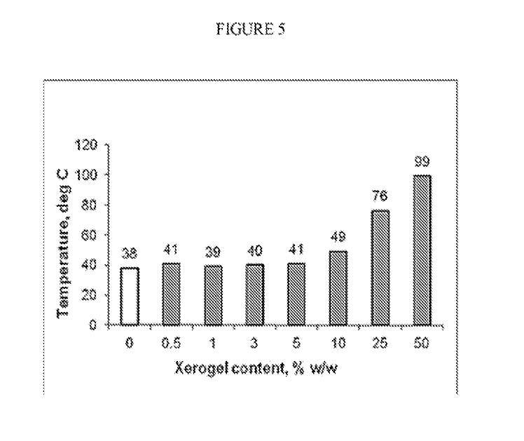

The glass transition temperature of the nanocomposites increases as the weight fraction of silica xerogel increases (FIG. 5). The Tg values for these nanocomposites do not scale linearly with the silica particle surface areas; that is, assuming spherical silica nanoparticles of constant density and volume directly proportional to the weight percentage in the nanocomposites, the calculated ratio of Tg/A, where A is the particle surface area, is not constant but rather decreases from 2.9 for the 5% xerogel nanocomposite to 1.5 for the 50% xerogel nanocomposite.

While the compositions and reaction conditions for forming the nanocomposites preclude covalent bonding between the copolymer chains and the silica, the observed Tg behavior with increasing xerogel content is consistent with an increasing number of interfacial non-covalent binding interactions including hydrogen bonding between silica-derived hydroxyl groups and the copolymer's PEG chain oxygen atoms and DTE amide group nitrogen atoms

### Mechanical Properties.

The Young's moduli of the nanocomposites exceeded by factors of 5 to 20 times those of the copolymers or of composites made with micron-scale silica particles. Increasing the fraction of xerogel in the nanocomposites increased the glass transition temperature and the mechanical strength but decreased the equilibrium water content, which were all indicative of strong non-covalent interfacial interactions between the copolymers and the silica nanoparticles.

The Young's moduli for all of the micron-scale xerogel particle composites and the nanocomposites were significantly greater than that of the poly(DTE-10% PEG1k)carbonate copolymer alone and increased with decreasing xerogel particle size (FIG. 6). At a fixed 30 wt % xerogel content, the composite with the smallest micron-scale particle size range (10-20 μm) had a modulus of 664 MPA compared to a modulus of 430 MPa for the composite with the larger particle size range (70-105 μm). In contrast, the modulus of the nanocomposite at this same 30 wt % xerogel concentration was greatly increased to 920 MPa. The Young's moduli for nanocomposites at silica xerogel concentrations between 0.5% and 5% were between 316 MPa and 384 MPa, about twice that of the poly(DTE-10% PEG1k) carbonate copolymer alone, and increase rapidly to 920 MPa at 30% xerogel (Table 1).

The elongation at fracture, measured here as (L−Lo)/Lo, where L is the final and Lo the initial sample lengths, for the nanocomposites was essentially the same as that of the copolymer, 1230%, for xerogel concentrations up to 3% but began to decrease above that concentration, diminishing to 5% at 30% xerogel, which is indicative of the brittle, inflexible nature of the nanocomposites at very high xerogel loadings. Similarly, the Young's modulus of nanocomposites based on poly(DTO-0% PEG Ilk carbonate) increased rapidly from 4.4 to 80 MPa when the silica loading was varied from 5 to 10 wt % (Table 1).

TABLE 1
Tensile properties of the poly(DTE-10% PEG1k carbonate) and
poly(DTO-10% PEG1k carbonate) nanocomposites depend on the
silica xerogel concentrations.

| | | | Yield | Ultimate |
|---|---|---|---|---|
| | Modulus, | Elongation at | strength, | tensile |
| Sample | MPa | fracture, % | MPa | strenght, MPa |
| E0010 | 167 ± 18 | 1230 ± 48 | 14 ± 2 | 44 ± 2 |
| E0010/N0.5 | 316 ± 15 | 1307 ± 61 | 26 ± 4 | 48 ± 2 |
| E0010/N1 | 379 ± 51 | 1139 ± 85 | 22 ± 5 | 37 ± 2 |
| E0010/N3 | 351 ± 42 | 1204 ± 14 | 22 ± 3 | 34 ± 1 |
| E0010/N5 | 384 ± 121 | 531 ± 57 | 19 ± 4 | 26 ± 2 |
| E0010/N25 | 768 ± 61 | 12 ± 4 | 48 ± 4 | 48 ± 4 |
| E0010/N30 | 920 ± 102 | 5 ± 1 | did not yield | 30 ± 4 |
| O0010 | 4 ± 0.3 | 777 ± 52 | 1.3 ± 0.1 | 15 ± 4 |
| O0010/N5 | 4.4 ± 0.5 | 531 ± 21 | 1.7 ± 0.3 | 48 ± 3 |

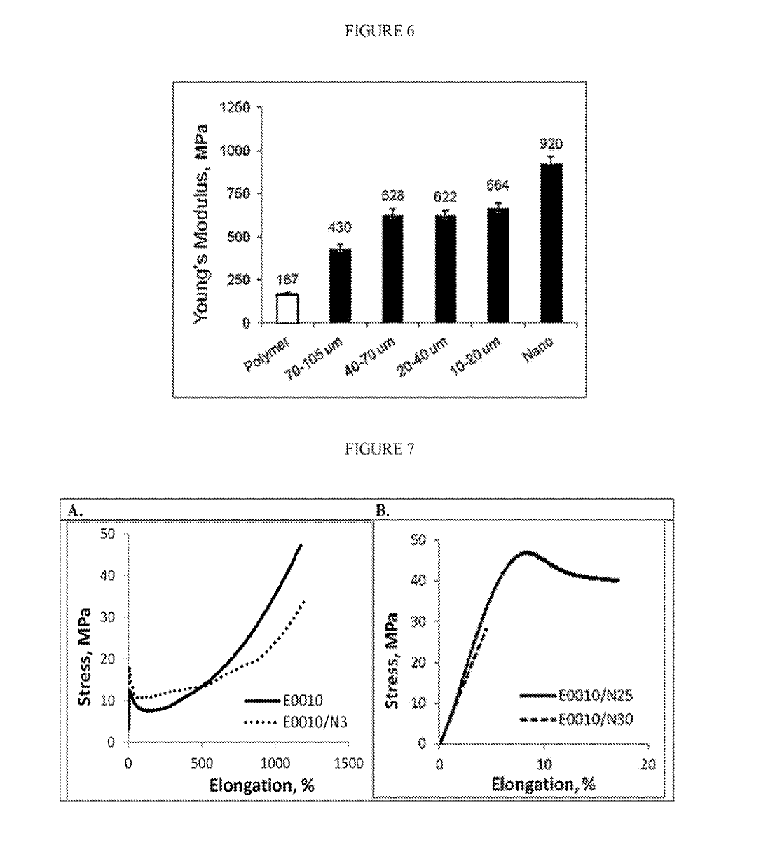

The tensile behavior of the nanocomposites changed gradually as the silica content increased. When small fractions of silica were present in the nanocomposites they became stronger but remained just as ductile as the copolymer alone (Table 1 and FIG. 7, top). From 0.5 to 5 wt % of silica loading, the stress at yield stayed almost constant at around 22 MPa, about 50% higher than that of the copolymer.

When the nano-silica fraction was increased (E0010/N25 in Table 1 and FIG. 7, bottom) the nanocomposite became much stronger as evidenced by the three-fold increase of the stress at yield compared to the copolymer (48 MPa and 14 MPa, respectively). However, the polymer chain movement under stress was restricted by the silica and the nanocomposite was much less ductile (only 12% elongation at fracture). As the silica loading reached 30 wt %, the nanocomposites became even less ductile and did not yield (E0010/N30 in FIG. 7, bottom).

### Equilibrium Water Uptake

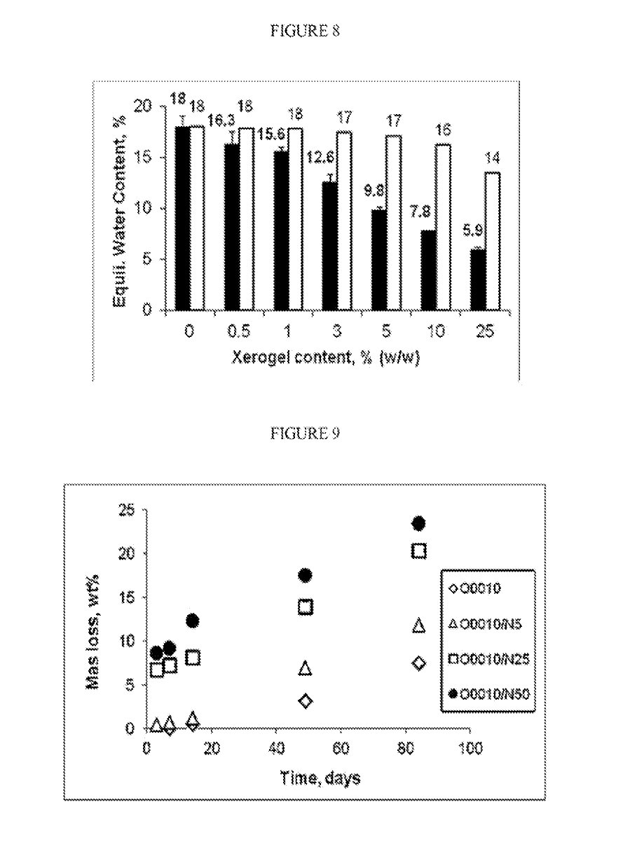

As the silica loading in the nanocomposite increased, the equilibrium water content (EWC) decreased (FIG. 8). The copolymer itself was a weakly absorbant hydrogel with an EWC of 18%. If the silica in the nanocomposites were inert and did not take up water or interact with the copolymer chains, then the amount of water uptake would be directly proportional to the mass fraction of copolymer present.

For example, for the 25% silica xerogel-containing nanocomposite, the mass fraction of copolymer in the nanocomposite was 75% and that mass of copolymer would by itself have an EWC of 13.5% (FIG. 8). However, the observed EWC for that nanocomposite was only 5.9%, which further demonstrated a significant interfacial interaction between the silica and the copolymer such that copolymer chain mobility was greatly restricted and water uptake as thereby reduced. For the microcomposites, the EWC was also found to decrease as the silica concentration increased, a trend that was seen with many but not all composites and which depended upon the nature of the polymers and inorganic components, their particle volume fraction and any non-covalent or covalent bonding between the components.

### Hydrolytic Degradation and Erosion.

The mass loss of the poly(DTE-10% PEG1k)carbonate copolymer by itself during incubation in buffer solution at 37° C. was negligible for 6 days and then increased slowly over several weeks (FIG. 9). The mass loss for the nanocomposites during incubation was significantly greater than that of the copolymer alone at every time interval and increased with increasing silica xerogel concentration. The kinetics of nanocomposite degradation after the initial 24 hr period were essentially linear with time and increased only slightly as the silica weight fraction increased.

Nanocomposite degradation was faster than for the copolymer alone, which was ascribed to the rapid dissolution of nano-scale silica particles, There was a significant mass loss in the first 24 hr for the nanocomposites of up to 8% for the 50% silica-containing specimen and, since the copolymer itself did not significantly degrade in that time frame, this mass loss of the nanocomposites was attributed to the rapid dissolution of the silica nanoparticles adsorbed on or near the outer surfaces of the specimens. The water uptake and degradation rate of the composites can be increased by increasing the hydrophilic PEG content of the copolymers and can be decreased by substitution of the more hydrophobic DTO monomer for the DTE monomer.

Drug Release from Nanocomposites

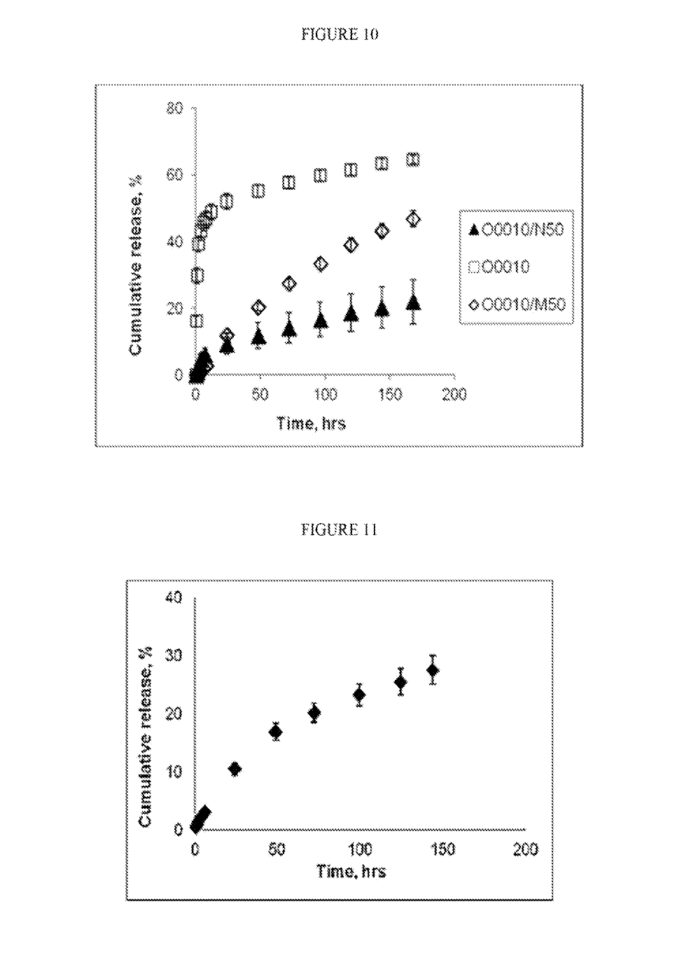

The release kinetics of bupivacaine from the microcomposites and nanocomposites were compared to the poly(DTO-10% PEG1k)carbonate copolymer alone at a fixed loading of 8 wt % bupivacaine. The copolymer alone exhibited an initial very large burst release stage of 50% of the drug load in the 24 hr followed by a second stage of relatively constant slow release thereafter (FIG. 10). The nanocomposite also exhibited 2-stage behavior but had a much reduced initial stage release of only 10% of the drug in the first 24 hours and thereafter continued to release the drug at a rate comparable to that of the copolymer.

The relatively small difference in the hydrolytic degradation of the copolymer and the nanocomposite over the first 24 hr cannot explain the significant reduction in the initial release from the nanocomposite. Rather, this difference was ascribed to differences in water influx and drug efflux from the samples that were determined by the different physical states of the co-polymer chains.

When no silica particles are present, the drug-loaded copolymer is in a rubbery state (the Tg is 2° C.). In contrast, the nanocomposites are in a glassy state as evidenced by the higher Tg (59° C.). Therefore the copolymer chain mobilities in the nanocomposites are more restricted and water uptake is reduced, which slows drug solubilization and diffusion out of the nanocomposite compared to the pristine copolymer. The silica nanoparticles appear also to impede efflux from the nanocomposites by binding the drugs and/or by acting as physical barriers to flow.

In contrast, in the microcomposites the drug is initially confined entirely within nanopores of the xerogel particles and the copolymer matrix acts as a barrier membrane to further control water influx and drug efflux. The porosity of the micron-scale xerogel particles and the hydrophobicity of the copolymer matrix determine the drug release kinetics of the microcomposites, which for the O0010/M50 is faster than for the nanocomposite and essentially zero-order, i.e., pseudo-zero order over the first 72 hr. (FIG. 10)

“Pseudo-zero order” release is a well-known term of art referring to a kinetic drug release profile equivalent to essentially zero order release obtained by balancing diffusional slow-down and acceleration of the release rate by erosion. For purposes of the present invention “essentially zero-order release” and “near zero-order release” refer to a drug release rate at or near zero order over the sustained release phase of drug delivery under physiological conditions. Compositions with drug release at or near zero order have drug release coefficients that are essentially unchanged relative to the arithmetic mean over the sustained release phase of drug delivery under physiological conditions.

For example, in one embodiment. “essentially zero-order release” and “near zero-order release” refer to the release kinetics of polymer compositions under physiological conditions, in which the release rate of drug from the composition varies by no more than ±10% over the sustained release phase following the initial burst for a period of about 1 week to about 4 years. One embodiment had a sustained release for a period between about one month to about three years. Additional embodiments included compositions in which the release rate of drug from the composition varied by no more than ±9%, ±7.5%, or ±5% over the sustained release phase following the initial burst.

As guided by the present specification, one of skill in the art can manipulate the release profile by adjusting certain features of the composition, for example, the polymer(s), drug(s), level of drug loading, surface area, etc. Furthermore, the initial burst can be shortened to less than one week by subsequent processing such as rinsing the blend to remove drug at or near the surface or by coating the composition with a bioerodible polymer that is either drug free or has a reduced drug content.

The release rate of the antibiotic, rifampicin, from the nanocomposite (FIG. 11) was similar to that of bupivacaine from the nanocomposite. The initial loading of rifampicin was 10% wt:wt in the poly(DTO-10% PEG1k) carbonate (O0010/N10) nanocomposite. The initial rifampicin release over the first 24 hr was about 10% of the rifampicin loading and this was followed by a slower second stage release rate.

This is consistent with the similar physical properties of the two drugs: rifampicin is hydrophobic, with an octanol/water partition coefficient of log P=2.72 and water solubility of 1.4 mg/ml; similarly, for bupivacaine, the log P is 3.41 and the water solubility is 2.4 mg/mi. When the cumulative rifampicin release data are plotted as a function of t^{1/2} they can be fit by a single straight line (correlation coefficient of 0.98) which is consistent with the Higuchi model for diffusion controlled drug release.

Thus, optically clear nanocomposites of tyrosine-PEG-derived polyether carbonate copolymers and silica xerogels were obtained by the simple process of mixing the silica sol into the copolymer solution. Based on TEM micrographs, the particle size of the silica xerogels was about 5 to about 50 nm homogeneously distributed throughout the copolymer matrix. Material properties of the nanocomposites depended on the amount of the silica xerogel present. Increasing amounts of nano-sized xerogel increased the Tg and the mechanical strength, but decreased the equilibrium water content (EWC). The increase in the Tg, and the fact that the experimental EWC's for various compositions were significantly lower than the theoretical values, is indicative of a strong interfacial interaction between the copolymer and the silica nanoparticles.

The hydrogen bonding interfacial interactions between the large number ethylene oxide units in the copolymer backbone and the silica nanoparticles can act as physical cross-linkers and explain the reduced polymer chain mobility reflected by the increased Tg. The Tg behavior of the present nanocomposites contrasts with similarly prepared nanocomposites based upon poly(ε-caprolactone) and TEOS-derived silica, where no significant increase in Tg is observed with increased silica content in the nanocomposites. The difference between the poly(ε-caprolactone) nanocomposites and poly(DTE-10% PEG1k) carbonate nanocomposites can be ascribed to the large number of PEG oxygen atoms present in poly(DTE-10% PEG1k) carbonate copolymers compared to the poly(ε-caprolactone), which provides only a very limited number of ester group oxygen atoms for hydrogen bonding to the silica-derived hydroxyl groups, and hence there is no significant increase in interfacial hydrogen bonding as the silica nanoparticle content is increased in the poly(ε-caprolactone) nanocomposites.

Sustained controlled delivery of two clinically important drugs, rifampicin and bupivacaine, was obtained from the drug-loaded nanocomposites. After a small initial burst release that could be attributed to the dissolution of the loosely bound drug, bupivacaine was released at a constant rate for a total of 7 days. The amount of rifampicin released from the nanocomposite in the first 24 hr was 0.06 mg/ml, which exceeds the minimum inhibitory concentration (MIC) for planktonic methicillin-resistant Staph. aureus (MRSA) infections and for Staph. epidermidis biofilms. The release of rifampicin from the nanocomposite was in accord with the Hugichi model (i.e., follows linear behavior when plotted as t^{1/2}) so higher drug loading levels would be expected to result in greater solution concentrations such that treatment of biofilms in vivo would be possible.

By varying the silica nanoparticle loading and the copolymer matrix compositions it has now been demonstrated that nanocomposites of silica xerogels and tyrosine-poly(ethylene glycol)-derived poly(ether carbonates) provide a broad, tunable range of mechanical properties and bio-degradability under physiological conditions. The strong tensile properties of the nanocomposites, and their controlled release of hydrophobic drugs make these biomaterials highly attractive for applications such implantable drug delivery depots and wound dressings for treating pain and orthopedic infection, for tissue engineering substrates, for cardiovascular stents and for nerve guides or conduits.

The polymers/copolymers and silica nanoparticles independently can contain therapeutic agents, are independently capable of binding these agents, and can independently release such agents. It is sufficient that either the polymer or the nanoparticles contains therapeutic agents, although both can contain them. The nanocomposite of the nanoparticles in the polymer provides a unique matrix that enables far better control of the kinetics of delivery of the therapeutic agents than can be attained by either the polymers or the nanoparticles alone. These nanocomposites provide unique control of binding and release of therapeutic agents, thereby providing controlled delivery of the therapeutic agents for healthcare applications. The nanocomposites combine the advantages of the drug binding and release kinetics of silica sol-gels with the mechanical flexibility and drug binding of polycarbonate films, and further, are uniquely formable into various devices.

The drug delivery system of the present invention permits fine tuning of drug loading and drug release kinetics while providing the mechanical strength and stability properties characteristic of heterogeneous nanocomposites. The nanocomposites of the present invention are designed to reduce burst release and provide the continuous and constant rates of release of a therapeutic agent that is essential for sustained, effective therapeutic activity. The release of one or more therapeutic agents from the present nanocomposites can be pseudo first order release (i.e., the release kinetics of the present nanocomposites can be characterized by a substantially constant release of therapeutic agent over time).

Conditions for synthesizing the silica nanoparticles can be controlled to produce a particular controlled release profile for a therapeutic agent corresponding to a concentration with known therapeutic effect. The drug molecules, incorporated in nano-sized pore channels of the nanoparticles and non-covalently bound by the copolymers of the biocompatible film, will release by diffusion through the aqueous phase that penetrates into the nanocomposites.

The parameters of the silica nanoparticle synthesis affects the fundamental properties of the particles that control release of the therapeutic agent. These parameters include specific surface area, granule or powder size, and pore size and porosity. Formation of nanocomposites of nanoparticles in polymers, such as in poly(DTR-co-PEG carbonate), can be by compression molding; the copolymer compositions (pendent ester R chain lengths, PEG molecular weight and PEG/DTR molar ratios) can be varied systematically to achieve an optimum loading efficiency of the drug-loaded silica sol-gel nanoparticles and to improve the mechanical properties of the films, such as tensile and flex strengths.

The nanocomposites of the present invention are useful in depot delivery of therapeutic agents such as organic drug compounds, genes, oligonucleotides, and proteins, and in wound treatment applications such as for compartment syndrome, chronic and phantom pain treatment, hemostasis, and infection control. The nanocomposites of the present invention can be useful in various therapeutic applications, including treatment of pain resulting from wounds and prophylactic treatment of compartment syndrome associated with wounds. For the treatment of pain, silica-based nanoparticles and tyrosine-based copolymers can be synthesized to effectively bind and release therapeutic agents such as bupivacaine and mepivacaine.

For the prophylactic treatment of compartment syndrome, sol-gels and copolymers can be synthesized to effectively bind and release anti-apoptotic and pro-angiogenic factors. While the therapeutic nanocomposites of the present invention can be described in connection with a single drug, it will be understood by those skilled in the art that the therapeutic nanocomposites are capable of concurrent delivery of multiple drugs.

### Pain Treatment

Also provided is a novel approach to the treatment of chronic pain arising from wounds with severe tissue damage and/or from surgical procedures. This approach entails controlled release of a selected local anesthetic from biocompatible, biodegradable nanocomposites applied directly to the wound site beginning as soon as possible after the wound or surgery occurs. The biocompatible nanocomposites provide sustained treatment of the peripheral nerves located at the wound site with a local anesthetic that functions as a sodium channel blocker to shut down the firing of the afferent axons that carry the pain signals back to the brain. This technology can potentially reduce or eliminate the imprinting process in the central nervous system that is recognized as a key component of chronic pain.

In accordance with this aspect of the invention, a local anesthetic can be bound to a nanocomposite matrix comprised of silica nanoparticles incorporated in a tyrosine based polycarbonate-PEG film to provide controlled release of the anesthetic. The local anesthetic is preferably mepivicaine or bupivicaine, because of their high activity with low cardiovascular side effects. The nanocomposites are preferably effective for up to 72 hours, permitting easy use on the battlefield, in combat support hospitals, and civilian and veterans' hospitals.

Bupivacaine and mepivacaine can be incorporated by addition of appropriate solutions to a mixture of the silica nanoparticles (silica sol) and the biodegradable, biocompatible copolymer, preferably a desaminotyrosyl tyrosine ester-PEG carbonate copolymer. The immediate and sustained delivery of local anesthetic enables quicker recovery times, shorter hospital stays, earlier achievement of physical therapy milestones, and lower rates of narcotic use and abuse among military and civilian patient populations.

### Prophylactic Treatment of Compartment Syndrome

In compartment syndromes, there is a zone of tissue that is between normal and irreversibly damaged, and in this zone anti-apoptotic and pro-angiogenic factors can be useful to restore function. Thus, in accordance with one aspect of the invention, provided is a prophylactic treatment of a wound site to avoid the onset of compartment syndrome and associated fasciotomy treatment. Even when fasciotomy is ultimately required, treatment in accordance with the invention provides for more rapid and complete healing of incision and wound sites.

In acute compartment syndrome, fluid accumulates and the intramuscular pressure (IMP) increases. Removal of only about 1 ml of interstitial fluid can result in a reduction of intramuscular pressure such that intramuscular pressure (IMP) is restored to a normal range. Thus, in accordance with one aspect of the invention, nanocomposites made from polymers such as tyrosine-based block copolymers and silica nanoparticles can be designed and formed as a polymer-nanoparticle wound dressing to remove fluid from injured muscle compartments.

The biocompatible nanocomposites can be composed of tyrosine-based copolymers and silica sol-gels in the form of nanocomposite films or other shaped devices that are adapted to absorb 100% or more of their weight in body fluid while maintaining their flexibility, adhesion, and mechanical integrity. To provide this type of fluid adsorption, well-established synthetic polymer chemistry methods for forming cross-linked polymers can be employed.

Further, the nanocomposite dressing is capable of concurrently delivering a selected therapeutic agent to the wound site. The therapeutic agent can be incorporated in the resorbable nanocomposite of silica nanoparticles and biodegradable, biocompatible copolymer. The therapeutic agent incorporated into the nanocomposite can include one or more of an anti-apoptotic factor, a pro-angiogenic factor, and a polymeric surfactant.

### Tyrosine-Based Polycarbonate-Poly(Ethylene Glycol) Copolymers

Degradable polyesters, poly(glycolic acid) (PGA), poly(lactic acid) (PLA), their copolymers (PLGA), and polydioxanone, are the predominant synthetic, degradable polymers with extensive regulatory approval histories in the USA. Although the utility of these materials as sutures and in a number of drug delivery applications is well established, these polymers cannot meet many of the material properties required for drug delivery devices.

For example, all of these polyesters release acidic degradation products, limiting their utility to applications where acidity at the implant site is not a concern. They also tend to be relatively rigid, inflexible materials, a disadvantage when mechanical compliance with soft tissue or blood vessels is required. Finally, the chemical properties of these polyesters is not substantially tunable, being limited to only a few combinations of fixed monomer structures, which limits thermodynamic and kinetic parameters that control drug binding and release.

The present invention encompasses a broad class of tunable, desaminotyrosyl tyrosine ester (DTR) diphenolic monomers that can be used to prepare polycarbonates and other polymer families. Among these polymers, tyrosine-derived polycarbonates have been studied most extensively and have been found to be tissue-compatible, strong, tough, hydrophobic materials that degrade slowly under physiological conditions. Further, it is preferable to use tyrosine-based block copolymers rather than polylactides because of the far greater tunability of the tyrosine-based blocks and because the polylactides are known to have inflammatory effects in vivo whereas the tyrosine-based copolymers do not. When these tyrosine-derived diphenolic monomers are copolymerized with blocks of poly(ethylene glycol) (PEG), a class of poly(ether carbonate)s is obtained that is elastomeric with remarkable tensile strengths and elongations.

### Examples

### Materials and Methods

### Materials

Tetraethoxysilane (TEOS) was purchased from Strom Chemicals, Newburyport, Mass. Pyridine 99% was purchased from Acros (MorrisPlains, N.J.). Poly(ethylene glycol) of molecular weight 1.000 (PEG1K) and bis(trichloromethyl)carbonate were purchased from Fluka (Milwaukee, Wis.). Methylene chloride HPLC grade and methanol HPLC grade were purchased from Fisher Scientific (Morris Plains, N.J.). Tetrahydrofuran (THF) high purity solvent stabilized with 250 ppm BHT was purchased from EMD (Gibbstown, N.J.). 2-propanol, bupivacaine hydrochloride, rifampicin, Dulbecco's phosphate buffer saline, acetonitrile HPLC grade and water solution containing 0.1% (v/v) trifluoroacetic acid for HPLC were purchased from Sigma Aldrich (Milwaukee, Wis.).

### Methods

### Copolymer Synthesis and Characterization.

### Synthesis of Poly(DTR-co-PEG Carbonate)

These copolymers are referred to as poly(DTR-co-fPEG M carbonate) where R represents the type of ester pendent chain, f represents the percent molar fraction of PEG units present within the backbone, and M represents the molecular weight of the PEG blocks. Thus, poly(DTE-co- 5% PEG1000 carbonate) refers to a copolymer prepared from the ethyl ester of desaminotyrosyl-tyrosine containing 5 mol % of PEG blocks of average molecular weight of 1000 g/mol. This molecular design provides tunability through three independent variables to enable optimization of materials properties (i) the pendent chain R, (ii) overall PEG content f, and (iii) length (molecular weight) M of the PEG block.

There are an enormous number of possible structures with this molecular design, including copolymers of poly(DTO-co-fPEG1000 carbonate), where f 0%, 10%, 40% and 70% to provide a range of hydrophobic-to-hydrophilic properties. DTO (i.e, the octyl ester) was selected because it has been identified as an ester having superior thermodynamic solubility parameter for binding hydrophobic drug molecules. Synthesis was performed by adding the DTO monomer and PEG to round bottom flasks containing methylene chloride and anhydrous pyridine. At room temperature, phosgene solution in toluene was added over 90 min to the reaction mixture with overhead stirring. Tetrahydrofuran (THF) was then added to dilute the reaction mixture to a 5% (w/v) solution.

The copolymer was precipitated by slowly adding the mixture into 10 volumes of ethyl ether. For further purification, copolymers with lower PEG content (<70% by weight) were redissolved in THF (5% w/v) and reprecipitated by slowly adding the polymer solution into 10 volumes of water. Copolymers with higher PEG content (70% by weight) were redissolved in THF (10% w/v) and reprecipitated by slowly adding the polymer solution into 10 volumes of isopropanol. In each case, the precipitated copolymer was collected and dried under vacuum.

The molecular weight of the copolymers can be controlled by the duration of the reaction and determined by gel permeation chromatography using THF as the solvent and using polystyrene standards. Chemical structure and polymer purity can be monitored by FT-IR, H-NMR, and C-NMR. The glass transition temperatures (T_{g}), crystallinity, and melting points of each copolymer can be determined by differential scanning calorimetry (DSC) and the decomposition temperature obtained by thermogravimetric analysis (TGA), with heating rates for both DSC and TGA of 10° C./min using an average sample size of 15 mg.

Polycarbonate copolymers of poly(ethylene glycol) (PEG) and desaminotyrosyl tyrosine esters (DTR) can be prepared by solution phosgenation as illustrated in FIG. 3. These copolymers have weight-average molecular weights up to about 200,000 and have symmetrical molecular weight distributions. To obtain structure-activity relationships, copolymers were prepared with either 5% PEG 1000 or 5% PEG2000 and different pendent ester chains (R=E (ethyl), B (butyl), H (hexyl), and O (octyl)). Also, the effect of PEG content was determined by preparing a series of poly(DTE-co-PEG1000 carbonate)'s with PEG content ranging from 1 mol % to 70 mol %. All of these copolymers were soluble in common organic solvents and those with high PEG content (70 wt %) were also soluble in water. Increasing the length of the hydrophobic pendent R chain lowers the glass transition temperature. Ts, in a linear fashion. The copolymers were observed to be thermally stable up to about 300° C.

The binding and release of organic drug compounds by the copolymers is a function of the hydrophobicity of the drug molecules as well as the hydrophobicity of the copolymer. The relative affinity of the copolymers for a drug can be predicted by their thermodynamic solubility parameters.

Organosilanes such as tetraethyoxysilane (TEOS) or tetramethoxysilane (TMOS) were used as the precursor molecules for the synthesis of the silica sol-gels via hydrolysis and condensation reactions. The hydrolysis reaction, which can be either acid or base catalyzed, replaces alkoxide groups with hydroxyl groups. Siloxane bonds (Si—O—Si) are formed during subsequent condensation. Alcohol and water are byproducts of the condensation reaction and evaporate during drying. Theoretically, the overall reaction is as follows:

nSi(OR)₄+2nH₂O→nSiO₂+4nROH

However, in reality, the completion of the reaction and the chemical composition of the resulting product depend on the excess of water above the stoichiometric H₂O/Si ratio of 2. A number of other sol-gel processing parameters (such as pH of the sol, type and concentration of solvents, temperature, aging and drying schedules, etc.) can also affect the composition, structure, and properties of the resulting product.

The poly(ether carbonate) copolymer used throughout this study was composed of desaminotyrosyl tyrosine ethyl ester (DTE) monomer and poly(ethylene glycol) (PEG) of molecular weight 1,000 Daltons (FIG. 1), which is referred to as poly(DTE-co-10% PEG1k carbonate) and abbreviated as E0010. Similarly, poly(DTO-10% PEG1k carbonate) contained desaminotyrosyl tyrosine octyl ester (DTO) monomer and PEG and is abbreviated as O0010. The two copolymers were synthesized following a previously reported method and their structure is illustrated in FIG. 1. The copolymer composition was confirmed by ¹H NMR (DMSO-d6, Varian VNMRS 400 MHz spectrometer) and Fourier transform infrared spectroscopy (FTIR) (Avatar 380 spectrometer, Thermo Nicolet). The number average (Mₙ) and weight average (M_{w}) molecular weights of the copolymer were determined by gel permeation chromatography (GPC; Waters Corp. 515 HPLC pump, 717 autosampler, 410 RI detector, and Empower 2 software) with 103 and 105 Angstrom gel columns (Polymer Laboratories/Agilent, Santa Clara, Calif.) in series, with THF as mobile phase and a flow rate of 1 ml min⁻. Calibration was based on polystyrene standards (Polymer Laboratories/Agilent).

### Xerogel Micron-Scale Particle Synthesis.

### Comparison Method.

Silica xerogels were prepared at room temperature via a one-step acid catalyzed sol-gel process using tetraethoxysilane (TEOS) as a precursor at a water:TEOS molar ratio of 10:1. Briefly, 1.0 M HCl was added to a mixture of water and TEOS at a 10:1 molar ratio to a final pH of 2.2. A clear sol formed after vigorous stirring for 20 min. The sol was cast into cylindrical polystyrene vials that were sealed and the sol allowed to gel and ate at 37° C. for 2 days. Subsequently the vials were opened and the gels were allowed to dry in an oven at 37° C. for 3-4 days until the gel weight became constant. The silica gel was dried, crushed into granules, sieved using nylon meshes and sorted according to their particle size in 10-20 μm, 20-40 μm. 40-70 μm and 70-105 μm diameters. Gas (N₂) adsorption/BET analysis (Autosorb-1, Quantachrome, Boynton Beach, Fla.) was used to determine the surface area, pore size and pore volume of xerogels that were first dried and outgassed at 50° C. for 20 h. Bupivacaine-containing xerogel microparticles were prepared by dissolving the drug in methanol and adding this directly to the acid catalyzed sol.

### Micron-Scale Particle Composites Fabrication.

### Comparison Method.

Microcomposites of the copolymer and micron-scale xerogel particles, referred to herein simply as “microcomposites”, were prepared via solution blending method. For a typical 500 mg sample of microcomposite, 350 mg E0010 copolymer was dissolved in 7 mL THF and 150 mg dry xerogel with the desired particle size was vigorously mixed in for 2 minutes. The slurry was then poured into a PTFE mold and the solvent was slowly evaporated over 48 h in the fume hood to yield a uniform film. The resulting film was dried under nitrogen flow for 24 h and in a vacuum oven at 50° C. for 24 h. The micron-scale silica particle composites were abbreviated as, e.g., E0010/X30(10), meaning a matrix of the copolymer E0010 containing 30% (wt:wt) silica xerogel (X) having a particle size of 10-20 μm.

### Nanocomposite Synthesis and Morphology.

The nanocomposites were prepared in situ by adding deionized water to TEOS in a 20 mL scintillation vial to obtain a water:TEOS molar ratio. Rs, of 6-10. The TEOS hydrolysis reaction was catalyzed by adding 1 N HCl to a final concentration of 0.35M HCl. The reaction mixture was stirred at room temperature for about 16 hrs to allow complete TEOS hydrolysis without allowing the silica polycondensation reaction to reach the gel point. Volumes of silica sol were transferred into small vials containing 10% solutions of poly(DTE-10% PEG1k carbonate) in glacial acetic acid. The silica sol volumes transferred and the copolymer amounts used were chosen such that the theoretical amount of SiO₂ formed after hydrolysis corresponded to 0.5, 1, 3, 5, 10, 25, 30 and 50 wt % SiO₂:copolymer in the final nanocomposites.

When the silica sols were added to the copolymer solutions, all of the samples remained transparent with no macroscopic phase separation or precipitation observed.

The nanocomposite solutions were then stirred for 5 minutes, poured into Teflon Petri dishes and dried under nitrogen flow overnight, and then placed in a vacuum oven at 40° C. for a total of 96 h. The nano-scale silica composites were abbreviated as, e.g., E0010/N30, meaning a nanocomposite (N) of E0010 copolymer with 30% (wt:wt) silica xerogel.

Transmission electron microscopy (TEM) of the nanocomposite films was performed by embedding them in a low viscosity epoxy resin and then cutting 50 nm thick samples using an ultramicrotome equipped with diamond knife. The thin sections were transferred to carbon-coated copper grids (200-mesh) and imaged in a JEOL 100CX transmission electron microscope operated at accelerating voltage of 100 kV. No heavy metal staining of sections prior to imaging was necessary.

### Drug-Containing Nanocomposites.

Drug-loaded nanocomposites were prepared by stirring the pre-hydrolized TEOS solution with the copolymer solution for 1 minute and adding appropriate volumes of a 2 mg/mL bupivacaine or rifampicin solution in methanol followed by vigorous mixing for another minute. The mixture was then poured into Teflon dishes, dried first under nitrogen flow and then in a vacuum oven for 96 h at room temperature to avoid any possible drug degradation.

### Thermal Properties.

Thermogravimetric (TGA) experiments were performed in air and the temperature was ramped from 25 to 600 deg C. at a 10 deg/min rate. The glass transition temperature (Tg) was determined by differential scanning calorimetry (2910 Modulated DSC, TA Instruments) on 10-15 mg samples. Specimens were sealed in aluminum pans and subjected to a heat-cool-reheat temperature program from -50 to 150° C. at a heating rate of 10° C./min. The glass transition temperatures were taken as the inflection points in the second heating scans of the DSC temperature program.

Tensile Properties.

Tensile properties of thin copolymer and composite film strips (30×5×0.20 mm) were tested according to ASTM standard D882-91 on a Sintech 5/D tensile tester. Measurements were done in dry state at room temperature, and also in water at 370(C after the samples were pre-incubated in PBS at the same temperature for 1, 3 and 5 days respectively. The results for Young's modulus, elongation (strain) and tensile strength were averaged over 3-4 replicates. The width (5 mm) and thickness of each of the specimens tested were averaged over three measurements in different parts of each specimen. The initial grip speed was 2 mm/min, allowing for a reliable measurement of the elastic modulus; the yield point was calculated based on the zero slope criterion.

Equilibrium Water Uptake

Rectangular 200 μm-thick samples of polymer and composites were immersed in PBS at 37° C. without shaking. The average sample size was 60 mg. At given time points the specimens were removed from the buffer, dried on a paper towel and weighted. The equilibrium water uptake (EWC) was calculated as the ratio of the mass gain during incubation and the weight of the dry specimen: EWC=(W_{wet}−W_{dry})/W_{dry}, and was taken as the point at which two consecutive time point samples had the same mass. Samples were run in triplicate.

In Vitro Degradation

For the accelerated hydrolytic degradation study, rectangular 200 μm-thick, 60 mg copolymer and nanocomposite samples were incubated in 20 mL PBS, pH 7.4 at 37° C. and mixed at 100 rpm. The buffer was replaced every two days to maintain sink conditions for the degradation products and the mass loss of these samples was followed for 90 days. For each of the nanocomposite formulations (0.5, 1, 3, 5, 10, 25, 30 and 50% by weight silica loading) and the copolymer alone, the samples were removed from the buffer at selected time intervals, rinsed with DI water, freeze-dried and weighted. The mass loss was calculated as the ratio between the mass loss during the incubation period and the weight of the sample before incubation.

In Vitro Drug Release and Antimicrobial Activity

The release rates of the local anesthetic, bupivacaine (BP), from the micron-scale particle composite and the nanocomposite films were measured for up to seven days using 30 mg of samples of the composites incubated in 6 mL PBS at 37° C. in a Julabo SW2 water bath shaker at 100 rpm. The incubation medium was completely withdrawn at specified time intervals and replaced with 6 mL fresh buffer. The withdrawn samples were diluted 1:1 (v/v) with acetonitrile and analyzed by high performance liquid chromatography (HPLC). All experiments were performed in triplicate. The same procedure was used for the determining the release rate of bupivacaine and rifampicin from a nanocomposite film. Validity of the method was established through a study of specificity, linearity and accuracy according to the International Conference on Harmonization (ICH) guidelines.

The antimicrobial activity of rifampicin and rifampicin-containing nanocomposites were determined against Staphylococcus aureus UAMS-1 (ATCC 49230), a clinical osteomyelitis strain, using a slightly modified Kirby-Bauer zone of inhibition (ZOI) method. Frozen S. aureus UAMS-1 stock was thawed and diluted in 4 ml Mueller-Hinton II broth (cation-adjusted) (MIHBII) to a density of 1 McFarland unit (˜0.25 AU), then used to streak a lawn of bacteria onto Mueller-Hinton agar plates. Three circular 6 mm diameter discs of each nanocomposite were placed on the agar plates equidistant from each other and midway between the center and edge of the plate. The plates were incubated overnight at 37 C., and the circular ZOI (absence of bacterial colonization, which was readily distinguished by visual inspection) measured with an electronic caliper.

Although the invention herein has been described with reference to particular embodiments, it is to be understood that these embodiments are merely illustrative of the principles and applications of the present invention, and are not intended to limit the invention in any way. It is therefore to be understood that numerous modifications can be made to the illustrative embodiments provided herein, and that other embodiments can be devised without departing from the spirit and scope of the present invention as defined by the following claims.

All references cited herein are incorporated by reference in their entireties.

1. A copolymer-xerogel nanocomposite comprising:
a biodegradable, biocompatible copolymer;
silica nanoparticles; and,
therapeutic agent.

2. The nanocomposite of claim 1, wherein the biodegradable, biocompatible copolymer has a molecular weight greater than 20,000 Daltons.

3. The nanocomposite of claim 1, wherein said therapeutic agent is an antibiotic, a local anesthetic, and or a combination thereof.

4. The nanocomposite of claim 1, wherein said therapeutic agent is rifampicin, bupivacaine, or both.

5. The nanocomposite of claim 1, wherein said biodegradable copolymer comprises a copolymer of tyrosine-poly(alkylene glycol)-derived poly(ether carbonate).

6. The nanocomposite of claim 5, wherein said biodegradable copolymer comprises a polycarbonate comprising desaminotyrosyl tyrosine ester and poly(ethylene glycol).

7. The nanocomposite of claim 1, wherein the nanocomposite provides controlled release of said therapeutic agent.

8.-10. (canceled)

11. A drug depot, wound dressing, cardiovascular stent, or tissue scaffold comprising the nanocomposite of claim 1.

12.-24. (canceled)

25. The nanocomposite of claim 1, wherein the nanocomposite provides pseudo first-order release of said therapeutic agent.

26. The nanocomposite of claim 1, wherein the silica nanoparticles have a diameter of no more than 50 nm.

27. The nanocomposite of claim 1, wherein the copolymer contains the therapeutic agent.

28. The nanocomposite of claim 27, wherein at least some of the silica nanoparticles contain the therapeutic agent.

29. The nanocomposite of claim 1, wherein at least some of the silica nanoparticles contain the therapeutic agent.

30. A method comprising applying to a subject or implanting within a subject a nanocomposite according to claim 1.
